(19) United States
(12) Patent Application Publication
(10) Pub. No.: US 2024/0131227 A1
(43) Pub. Date: Apr. 25, 2024

(54) MULTIFUNCTIONAL SEALING MEMBER FOR IMPROVED ENDOTHELIALIZATION AND PARAVALVULAR LEAKAGE

(71) Applicant: EDWARDS LIFESCIENCES CORPORATION, Irvine, CA (US)

(72) Inventors: Ekaterina Tkatchouk, Huntington Beach, CA (US); Oleksandr Barannyk, Irvine, CA (US); Jeanette Jasmine Corona Kelly, Anaheim, CA (US)

(21) Appl. No.: 18/541,795

(22) Filed: Dec. 15, 2023

Related U.S. Application Data
(63) Continuation of application No. PCT/US2022/033461, filed on Jun. 14, 2022.
(60) Provisional application No. 63/211,384, filed on Jun. 16, 2021.

(51) Int. Cl. A61L 27/52; A61L 27/18; A61L 27/26; A61L 27/34

(52) U.S. Cl. CPC A61L 27/52; A61L 27/18; A61L 27/26; A61L 27/34; A61L 2430/20

(57) ABSTRACT

A sealing member for an implantable prosthesis includes a substrate comprising a substrate material, and one or more hydrogel structures disposed on an outwardly facing surface of the substrate. The hydrogel is stimulus responsive, and undergoes a change in volume, stiffness, or both when exposed to the stimulus. The sealing member promotes endothelialization and/or reduces paravalvular leakage.

### CROSS-REFERENCE TO RELATED APPLICATION

This application is a continuation of PCT patent application no. PCT/U20S22/033461, filed on Jun. 14, 2022, which claims the benefit of U.S. Provisional Patent Application No. 63/211,384, filed Jun. 16, 2021, each of which is incorporated herein in its entirety by this specific reference.

### FIELD

The present disclosure concerns embodiments of a multifunctional, stimulus-responsive sealing member for an implantable prosthesis.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Current implantable prostheses, such as bioprosthetic transcatheter and surgical heart valves typically are constructed with textile polyester, specifically polyethylene terephthalate as a sealing member (valve skirt). Polyethylene terephthalate, or PET is a strong, stiff synthetic fiber with excellent fatigue and creep resistance. One of the limits of PET based textile materials is their roughness due to yarn crossing and surface discontinuities. It is known that PET textiles induce standard foreign body reaction (FBR). A foreign body reaction (FBR) is a cell mediated response to a foreign material within biological tissue. It usually leads to fibrotic tissue overgrowth or encapsulation, pannus formation and calcification as a last step. FBR causes encapsulation of the skirt with fibrotic tissue and pannus overgrowth which spreads further with time and can obstruct movement of the leaflets of the prosthetic valve. FBR is largely influenced by the PET surface morphology and chemistry. Additionally, the skirt porosity might accelerate calcification and fibrotic tissue ingrowth. This behavior will depend on the size and number of the pores as well as on the morphology of the yarns that are involved in the textile construction. Another problem with known fabric skirts is that they typically are constructed of plain fabric weaves that may not form an adequate seal against the surrounding native annulus to sufficiently inhibit paravalvular leakage (PVL) in all patients.

Designing a novel synthetic non-hemolytic material with the ability to not trigger FBR and promote endothelialization instead is a very attractive solution to the existing challenges. If the material doesn't activate FBR, usually a healthy endothelial layer is formed on top of the implanted material. This endothelial layer provides dynamic control over homeostasis, influencing and preventing thrombosis and cell proliferation that can lead to regeneration and healthy tissue healing. One strategy to improve endothelialization is to use biodegradable polymeric materials with a chemistry that recruits endothelial and endothelial progenitor cells. It is widely accepted that surface morphology in the micro- or nanometer range affects cell adhesion, and modulates cell-cell interaction and cellular functions. Previously reported in vitro and in vivo testing of the effects of topography suggest that specific surface chemistry and porosity encourage endothelialization and promote anticoagulation. Skirt surface optimization in order to accelerate endothelialization, which is considered the best anti-hemolytic, anti-thrombotic and anti-inflammatory solution is highly beneficial.

### SUMMARY

This disclosure concerns embodiments of a smart, multifunctional sealing member for use in implantable prostheses, such as in the form of an outer or inner skirt mounted on the frame or stent of the prostheses. Methods of making the sealing member also are disclosed. The sealing member includes a material substrate and one or more hydrogel structures on an outer surface of the material substrate. “Smart” means that the hydrogel is stimulus responsive. For example, the hydrogel structure may undergo stimulus-induced changes in volume, stiffness, hydrophobicity, or any combination thereof. In some embodiments, the stimulus is a temperature change, a pH change, an ionic strength change, a solvent composition change, application of an electric field, application of a magnetic field, application of ultrasound, exposure to light, or any combination thereof. In any of the foregoing or following embodiments, the hydrogel may be a natural hydrogel, a synthetic hydrogel, or a combination thereof. In some embodiments, the sealing member is an outer skirt, and the hydrogel structure is an annular hydrogel ring.

In any of the foregoing or following embodiments, the hydrogel structure, in an ex vivo environment, may have a first non-expanded thickness T1 measured from the outwardly facing surface of the annular substrate to an outer surface of the annular hydrogel ring. The hydrogel structure, in an in vivo environment, has a second expanded thickness T2, wherein T2 is greater than T1.

In some embodiments, the sealing member comprises a plurality of spaced-apart hydrogel structures disposed on the outwardly facing surface of the substrate. The plurality of structures creates a labyrinth seal when the hydrogel expands in vivo.

In one aspect, a prosthetic heart valve includes an annular frame configured to be radially compressible and expandable between a radially compressed state and a radially expanded state; a valvular structure disposed within the frame and configured to regulate the flow of blood through the frame in one direction; and a sealing member comprising a fabric substrate and a hydrogel structure attached to the fabric structure, the hydrogel structure comprising a stimulus-responsive hydrogel and having an exposed outer surface configured to seal against tissue surrounding the prosthetic heart valve when implanted in a patient's body.

In one aspect, a sealing member for an implantable prosthesis comprises a substrate comprising a substrate material, the substrate having an outwardly facing surface and an inwardly facing surface; and a stimulus-responsive hydrogel disposed on the outwardly facing surface of the substrate.

In one aspect, a sealing member for an implantable prosthesis comprises an outer skirt, the outer skirt comprising a substrate comprising a substrate material, the substrate having an outwardly facing surface and an inwardly facing surface, the substrate having a length L; and a hydrogel structure disposed on the outwardly facing surface of the substrate, the hydrogel structure comprising a stimulus-responsive hydrogel.

A method for making the disclosed sealing member includes providing a substrate comprising a substrate material, the substrate having an outwardly facing surface and an inwardly facing surface; and forming a hydrogel structure on the outwardly facing surface of the substrate.

In some embodiments, forming the hydrogel structure comprises (i) extruding or molding the hydrogel onto the outwardly facing surface of the substrate to form the hydrogel structure; or (ii) mechanically attaching the hydrogel structure to the outwardly facing surface of the substrate; or (iii) chemically attaching the hydrogel structure to the outwardly facing surface of the substrate; or (iv) dip-coating or spray-coating a hydrogel layer onto the outwardly facing surface of the annular substrate, and molding or etching the hydrogel layer to form the hydrogel structure. The method may further include forming a plurality of spaced-apart hydrogel structures on the outwardly facing surface of the substrate.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an exemplary transcatheter prosthetic heart valve, according to one embodiment.

FIG. 2 is a perspective view of an exemplary transcatheter heart valve including a multifunctional outer skirt according to one embodiment.

FIG. 3 is a cross-sectional diagram showing one embodiment of a multifunctional sealing member.

FIG. 4 is a cross-sectional diagram showing another embodiment of a multifunctional sealing member.

FIG. 5 is a front view diagram showing yet another embodiment of a multifunctional sealing member.

FIG. 6 is a front view diagram showing still another embodiment of a multifunctional sealing member.

FIG. 7 a front view diagram showing another embodiment of a multifunctional sealing member.

FIG. 8 is a schematic representation of a stimulus-responsive polymer.

FIG. 9 shows chemical structures of exemplary thermoresponsive polymers.

FIG. 10 is a perspective view of a prosthetic valve having an outer skirt including a material substrate coated with a stimulus-responsive hydrogel as disclosed herein.

FIG. 11 is a perspective view of a prosthetic valve having an outer skirt including a material substrate and a plurality of annular hydrogel rings on an outer surface of the material substrate according to one embodiment.

### DETAILED DESCRIPTION

Smart, multifunctional sealing members disclosed herein comprise a material substrate and one or more hydrogel structures on an outer surface of the material substrate. “Smart” means that the hydrogel is stimulus responsive. For example, the hydrogel structure may undergo stimulus-induced changes in volume, stiffness, hydrophobicity, or any combination thereof. Embodiments of the disclosed multifunctional sealing members induce and promote re-endothelialization independently of substrate textile configuration. Advantageously, the multifunctional sealing member also reduces or eliminates paravalvular leakage.

### I. Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0).

In order to facilitate review of the various embodiments of the disclosure, the following explanations of specific terms are provided:

AA: acrylic acid

AAM: acrylamide

Annular: Ring-shaped.

Annulus: A ring-like structure; the base of a heart valve that supports the valve leaflets.

Biodegradable: As used herein, the term biodegradable means capable of being decomposed or broken down within the body.

Biosorbable: As used herein, the term biosorbable (or bioabsorbable) means capable of being dissolved and absorbed by the body.

Biostable: As used herein, the term biostable means remaining chemically stable within the body.

Bonded: As used herein, the term bonded means the hydrogel is bound to the substrate surface by other than mechanical means. For example, the hydrogel may be bound by covalent, ionic, or van der Waals-type interactions.

CHOL: cholesterol

Co-block polymer: A polymer formed from polymerization of two different monomers A and B, wherein the polymer chain includes homopolymer blocks of monomer A and blocks of monomer B in a linear sequence, e.g., AAAAA-BBBBBB.

Copolymer: A polymer formed from polymerization of two or more different monomers.

DMAEM: 2-(dimethylamino)ethyl methacrylate

DPPC: ,2-diplamitoyl-sn-glycero-3-phosphatidylcholine

DPPE: dipalmitoyl-sn-glycero-3-phosphatidylethanolamine

DPPG: dipalmitoyl phospatidylglycerol

DPTAP: 1,2-dipalmitoyl-3-trimethylammonium-propane

DSPC: 1,2-distearoyl-sn-glycero-phosphocholine

DSPE: 1,2,-distearoyl-sn-glycero3-phosphoethanolamine

HSPC: hydrogenated soy phosphatidylcholine

Hydrogel: A cross-linked three-dimensional network of polymeric chains that are capable of absorbing and retaining molecules (e.g., water, polar solvents, non-polar solvents, drugs in liquid form, or the like) in their three-dimensional networks. Hydrogel-forming polymeric chains comprise one or more hydrophilic functional groups in their polymeric structures, such as amino (NH₂), hydroxyl (OH), amide (—CONH—, —CONH₂), sulfate (—SO₃H), or any combination thereof, and can be natural-, or synthetic-polymeric-based networks.

Hydrolyze: Decompose by reaction with water. Hydrolysis of large molecules, e.g., polymers, can be partial or complete.

MMA: methyl methacrylate

Monomer: A molecule or compound, usually containing carbon, that can react and combine to form polymers.

mPEG2000: poly(ethylene glycol) methyl ether, average molecular weight, Mₙ, ˜2000

MPPC: monopalmitoyl phosphatidylcholine

MSPC: 1-stearoyl-2-hydroxy-sn-glycero-3-phosphocholine

NHMAAM: N-(hydroxymethyl) acrylamide)

Nitrocatechol:

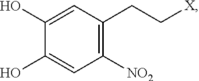

where X is —NH₂, —NHR, —OR, —NHCOR, —NHCOOR, or —NHCONHR, where R is aliphatic.

N,NDMAM: N,N-dimethylacrylamide

PBMA: poly(butyl methacrylate)

PEG: poly(ethylene glycol)

PEG2000: poly(ethylene glycol), average molecular weight, Mₙ, ˜2000

PEO: poly(ethylene oxide)

PLA: poly(lactide) or poly(lactic acid)

P(La-co-CL): poly(lactide-co-caprolactone)

PLGA: poly(D,L-lactide-co-glycolide)

PmDEGMA: poly(methoxydiethylene glycol methacrylate

P(MOEGA-DMDEA): poly[monomethyl oligo (ethylene glycol)acrylate-2-(5,5-dimethyl-1,3-dioxan-2-yloxy) ethyl acrylate]

PmTEGMA: poly(methoxytriethylene glycol methacrylate

PNIPAM: poly(N-isopropylacrylamide)

Polymer: A molecule of repeating structural units (e.g., monomers) formed via a chemical reaction, i.e., polymerization.

PPO: poly(propylene oxide)

Subject: An animal (human or non-human) subjected to a treatment, observation, or experiment.

Thermoplastic: Refers to a plastic that is capable of being heated and softened multiple times.

Thermoset: Refers to a plastic that can be heated and shaped only once.

TPU: Thermoplastic polyurethane

Triblock copolymer: A polymer formed from polymerization of different monomers, wherein the polymer chain includes three homopolymer blocks in a linear sequence, e.g., AAAAA-BBBBBB-AAAAA.

### II. Smart Multifunctional Sealing Member Design

Conventional textile sealing members, such as polyethylene terephthalate (PET), skirts induce standard foreign body reaction (FBR), accelerate calcification, and accelerate fibrotic tissue ingrowth. Surface chemistry and morphology control the type of cells that attach to the textile substrate. Surface porosity and hydrophilicity have emerged as tools to control and influence endothelial cell adhesion, migration, and proliferation.

Embodiments of smart, multifunctional sealing members are disclosed. In some embodiments, the multifunctional sealing member promotes improved and accelerated endothelialization, reduces or eliminates paravalvular leakage, or both. Advantageously, embodiments of the disclosed multifunctional sealing member also reduce FBR compared to conventional sealing members. Embodiments of the disclosed multifunctional sealing members comprise a material substrate and one or more hydrogel structures on an outer surface of the material substrate. The sealing member is referred to as a “smart” sealing member because its dimensions and/or properties may be altered by applying an environmental stimulus, such as a temperature change, a pH change, an ionic strength change, a solvent composition change, irradiation with light, exposure to ultrasound, application of an electric field, application of a magnetic field, or any combination thereof. The hydrogel structure undergoes a stimulus-induced change in volume, stiffness, hydrophobicity, or any combination thereof.

In any of the foregoing embodiments, the multifunctional sealing member can be used as a component of an implantable medical device. In some embodiments, the implantable medical device comprises a prosthetic heart valve and the sealing member is an outer skirt. In certain embodiments, the implantable medical device is a surgically implantable prosthetic heart valve for replacing any of the native heart valves (the aortic, mitral, tricuspid, and pulmonary valves). In other embodiments, the implantable medical device is a transcatheter prosthetic heart valve for replacing any of the native heart valves. Exemplary patents and publications relating to prosthetic heart valves in which embodiments of the disclosed sealing members may be useful include U.S. Pat. Nos. 7,993,394; 8,252,051; 8,454,685; 8,568,475; 9,393,110; 9,636,223; 9,662,204; 9,974,650; 9,974,652; 10,195,025; 10,226,334; 10,363,130; 10,413,407; 10,426,611; 10,433,958; 10,433,959; US 2018/0028310; US 2019/0167422A1; and WO 2018/222799, each of which is incorporated herein by reference in its entirety. In other embodiments, the implantable medical device can be a stent graft (e.g., a stent graft configured for implantation in the aorta) comprising a stent and the multifunctional sealing member mounted on the stent or a vascular plug comprising a plug and the multifunctional sealing member mounted on the plug.

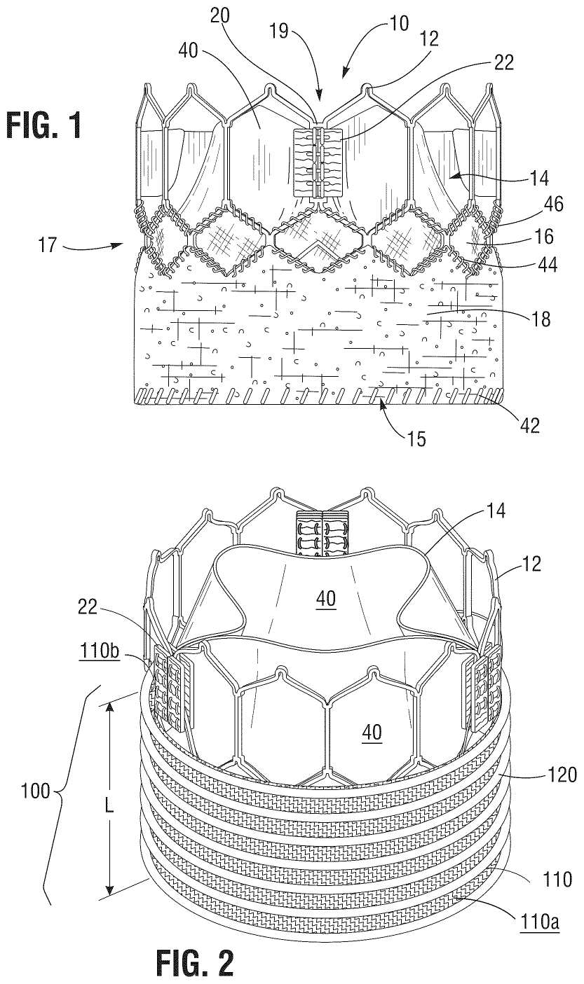

FIG. 1 shows a known transcatheter prosthetic heart valve 10, according to one embodiment, configured to be implanted via catherization, as known in the art. The illustrated prosthetic valve is adapted to be implanted in the native aortic annulus, although in other embodiments it can be adapted to be implanted in the other native annuluses of the heart (e.g., the pulmonary, mitral, and tricuspid valves). The prosthetic valve can also be adapted to be implanted in other tubular organs or passageways in the body. The prosthetic valve 10 can have four main components: a stent or frame 12, a valvular structure 14, an inner skirt 16, and a paravalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12 while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other embodiments there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape, and can be secured to the inner skirt 16 by sutures (not shown).

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., nickel titanium alloy (NiTi), such as nitinol), as known in the art. In some embodiments, when constructed of a plastically-expandable material, the frame 12 (and thus the prosthetic valve 10) can be crimped to a radially collapsed configuration on a delivery catheter and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel, a biocompatible, high-strength alloys (e.g., a cobalt-chromium or a nickel-cobalt-chromium alloys), polymers, or combinations thereof. In particular embodiments, frame 12 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

The inner and outer skirts 16, 18 can be formed from any of various suitable synthetic fabrics, such as polyethylene terephthalate (PET), and can comprise, for example, a braided, woven, or knitted fabric. The skirts 16, 18 can be attached to the frame 12 by stitching each skirt to struts of the frame. For example, the inflow edge of the outer skirt 18 can be attached to the frame with sutures forming stitches 42 that extend through the skirt and around struts at the inflow end of the frame. The outflow edge of the outer skirt 18 can be attached to the frame with sutures forming stitches 44 that extend through the skirt and around adjacent struts of the frame. Similarly, the inner skirt 16 can be attached to the frame with stitches 46 along the outflow edge of the skirt 16 and with stitches along the inflow edge of the skirt (not shown).

FIG. 2 is a perspective view of a prosthetic heart valve 90 that includes a multifunctional sealing member 100 in the form of an outer skirt in lieu of the outer skirt 18, according to one embodiment. Apart from replacing the skirt 18 with the sealing member 100, the prosthetic heart valve 90 can include any or all of the features of the prosthetic heart valve 10. Thus, components or features of the prosthetic heart valve 10 of FIG. 1 that can be incorporated in the prosthetic heart valve 90 are given the same reference numbers in FIG. 2 and are not further described for sake of brevity.

The sealing member, or skirt, 100 comprises a substrate 110 comprising a substrate material and having an outwardly facing surface 110a, an inwardly facing surface 110b, and a length L. In the embodiment of FIG. 2, the substrate 110 is an annular substrate. At least one hydrogel structure 120 in the form of an annular hydrogel ring is disposed on the outwardly facing surface 110a of the annular substrate 110. The hydrogel structure comprises a stimulus-responsive hydrogel. The hydrogel is not enclosed within a casing or covering and therefore has an exposed outer surface that can come into direct contact with body fluids (e.g., blood) and directly contact surrounding tissue of the native valve annulus when the prosthetic heart valve is implanted.

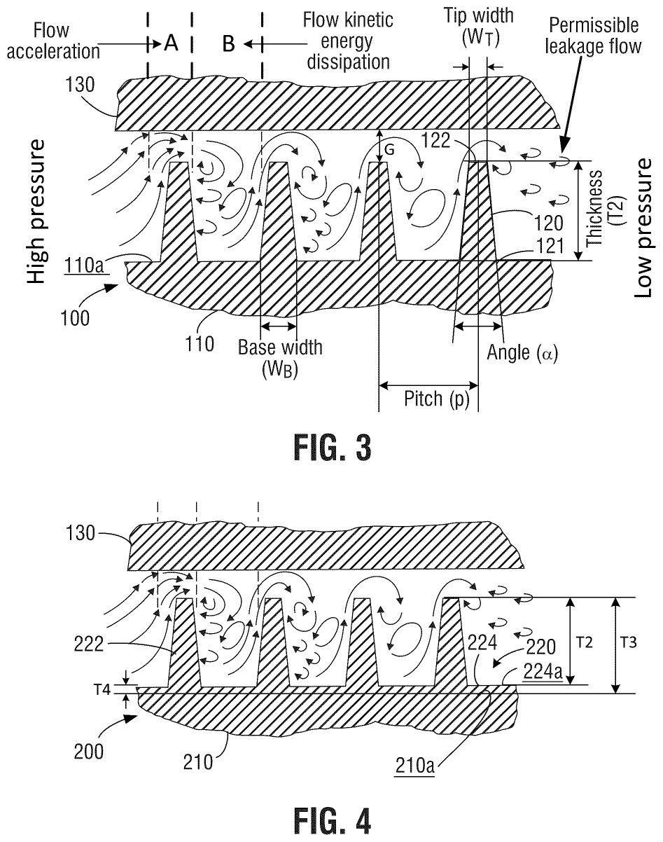

In some embodiments, as shown in FIG. 3, the hydrogel structure 120 has a base 121 in direct contact with the outwardly facing surface 110a and a tip 122. The hydrogel may be bonded or otherwise attached to the outwardly facing surface 110a. The exposed tip, or outer surface, 122 of the hydrogel structure is configured to seal against tissue 130 surrounding the prosthetic heart valve when implanted in a patient's body. In some implementations, such as the implementation exemplified in FIG. 2, the multifunctional sealing member 100 includes a plurality of spaced-apart hydrogel structures 120 in the form of spaced-apart annular hydrogel rings on the outwardly facing surface 110a of the substrate 110. For example, the multifunctional sealing member 100 may include from 2-20 annular hydrogel rings, such as from 2-15, 2-10, or 5-10 annular hydrogel rings. The hydrogel structures 120 in the illustrated example extending circumferentially around the substrate 110 and are spaced apart from each other along the length L of the sealing member.

The sealing member 100 can be attached or mounted to the frame 12 using one or more sutures to form stitches that extends through the sealing member and around struts of the frame, such as in the same manner as shown in FIG. 1. Hydrogels are relatively soft and frangible and alone may not be able to resist tearing or pull through of the stitches. On the other hand, as described in detail below, the substrate 110 can comprise a tough and tear resistant material, such as a woven, braided or knitted fabric. In this manner, the substrate 110 can resist pull through or tearing caused by the sutures to ensure a secure connection between the sealing member 100 and the frame 12. Thus, the sealing member 100 effectively combines the benefits of hydrogel material (e.g., improved and accelerated endothelialization and enhanced paravalvular valvular leakage prevention) and medical fabrics (e.g., tear-resistance).

FIG. 3 is a cross-sectional schematic diagram of a multifunctional sealing member 100 implanted within native tissue 130, which can represent an annulus of a native heart valve (e.g., the native aortic valve annulus) or a blood vessel (e.g., the pulmonary artery). The multifunctional sealing member 100 comprises a substrate 110 and a plurality of hydrogel structures 120 disposed on an outwardly facing surface 110a of the substrate 110. The hydrogel structures comprise a stimulus-responsive hydrogel.

In an unexpanded state (not shown), the hydrogel structure has a first non-expanded thickness T1 measured from the base to the tip of the hydrogel structure. The hydrogel structures 120 depicted in FIG. 3 are in an expanded state and have an expanded thickness T2 measured from the base 121 to the tip 122 of the hydrogel structure 120. The expanded thickness T2 is greater than the non-expanded thickness T1. A small gap G may remain between the tip 122 and the native tissue 130. In some embodiments, the thickness T2 may be from 1.1×T1 to 5×T1, such as from 1.2×T1 to 2×T1, from 1.3×T1 to 1×T1, or from 1.5×T1 to 1.7×T1.

The hydrogel structure base 121 has a width W_{B}, wherein W_{B} is less than the length L of the substrate 110. Each hydrogel structure 120 also has a top width W_{T} at the tip 122, where W_{T} is less than or equal to the base width W_{B}. In some implementations, the top width W_{T} is from 0.2 mm to 2 mm, such as from 0.2 mm to 1 mm, or from 0.5 mm to 1 mm In some embodiments, the hydrogel structure has a taper angle a from its base to the tip 122, wherein the taper angle a is from 0 degrees to 20 degrees. In certain implementations, the taper angle α is from 5 degrees to 20 degrees, or from 10 degrees to 20 degrees. In any of the foregoing or following embodiments, the expanded thickness T2 of the hydrogel structure may be related to the top width. In some embodiments, the expanded thickness T2 is from 10×W_{T} to 12×W_{T}.

When the multifunctional sealing member 100 includes a plurality of hydrogel structures 120, the structures have a center-to-center spacing, or pitch p, where p is related to the top width of the tip 122. In some embodiments, the pitch p is from 10×W_{T} to 18×W_{T}, such as from 12×W_{T} to 15×W_{T}. In the illustrated embodiment, the pitch and spacing between structures is constant along the entire length L of the skirt. In alternative embodiments, the pitch p and the spacing between the rings can vary along the length of the skirt. For example, the hydrogel structures may be grouped closer together in one more regions (e.g., near the top, middle, or bottom of the substrate), and spaced farther apart in other regions.

In some embodiments, as shown in FIG. 4, a multifunctional sealing member 200 comprises a substrate 210 comprising a substrate material, the substrate having an outwardly facing surface 210a. A hydrogel layer 220 is in contact with the outwardly facing surface 210a. In some embodiments, the hydrogel layer 220 completely covers, or substantially completely covers, the outwardly facing surface 210a. The hydrogel layer 220 has regions of a first average thickness T3 defining a plurality of spaced-apart hydrogel structures 222 alternating with regions 224 of a second average thickness T4. The first average thickness T3 is greater than the second average thickness T4. In an expanded state, the first average thickness T3 may be defined as the sum of the second average thickness T4 and the expanded thickness T2 of the hydrogel structures. The top width W_{T}, taper angle α, and pitch p, are as previously discussed. The base width W_{B} of the hydrogel structures 222 may be measured at a level corresponding to an outwardly facing surface 224a of the regions 224. Similar to the embodiment of FIG. 3, the pitch p and the spacing between the rings 222 can be constant along the length of the substrate or can vary along the length of the substrate.

In any of the foregoing or following embodiments, as shown in FIGS. 3 and 4, the expanded spaced-apart hydrogel structures may provide a labyrinth seal with the native tissue 130. The labyrinth seal provides a contorted path to inhibit leakage by providing a series of restrictions created by the hydrogel structures 120, with volumes of space between pairs of adjacent hydrogel structures. With reference to the numbering of FIG. 3, the gap G between each tip 122 and the native tissue 130 provides acceleration of flow through the gap G, resulting in isentropic expansion as the fluid exits region A, followed by dissipation of flow kinetic energy in the region B between adjacent hydrogel structures 120. A pressure drop occurs across each gap G, gradually reducing pressure, as shown in FIG. 3, and reducing PVL. The labyrinth seal and resulting pressure drop reduces leakage flow to a permissible level.

It should be understood that FIGS. 3 and 4 are schematic representations of an implanted sealing member. A native heart valve annulus typically is not perfectly circular and can vary in shape and diameter in the circumferential and axial directions (the axial direction being parallel to the direction of blood flow). Thus, once implanted, one or more of the hydrogel structures 120, 220 may fully or partially contact the surrounding tissue such that there is no gap G between a ring 120, 220, or a gap G only along a portion of the hydrogel structure 120, 220. Direct contact between a ring and the surrounding tissue can block the flow of PVL at that location. Thus, FIGS. 3 and 4 are intended to illustrate that where a gap G exists, the arrangement of hydrogel structures 120, 220 along the length of the substrate creates a pressure drop that prevents or minimizes PVL to an acceptable level.

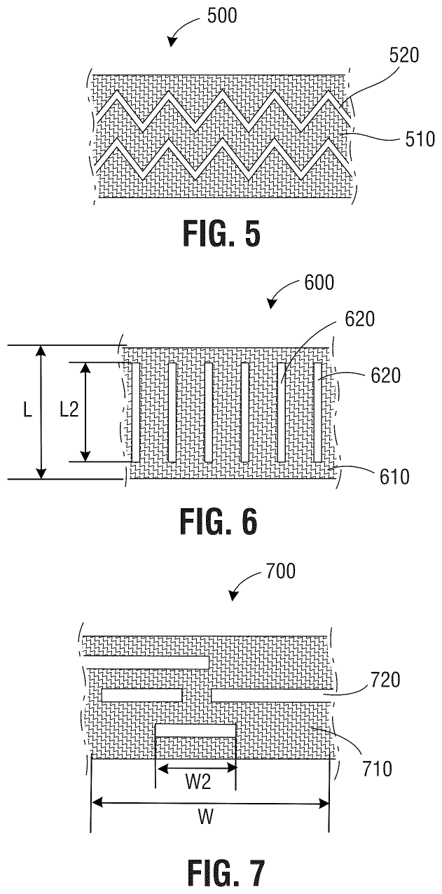

It should be understood that while FIG. 2 depicts a sealing member 100 comprising an annular substrate 110 with a plurality of hydrogel structures 120 in the form of annular hydrogel rings, other configurations are encompassed within the scope of this disclosure. FIGS. 5-7 are schematic diagrams showing three alternative hydrogel structures configurations.

FIG. 5 shows a sealing member 500 comprising a substrate 510 and a plurality of hydrogel structures 520 spaced apart from each other along the length L of the substrate. Each hydrogel structure 520 comprises a plurality of linear segments arranged in a zigzag configuration. When the substrate 510 is an annular form placed around the frame of a prosthetic heart valve, the hydrogel structures 520 are annular rings having a zigzag configuration.

FIG. 6 shows a sealing member 600 comprising a substrate 610 and a plurality of hydrogel structures 620 spaced apart from each other along the circumference of the substrate. The hydrogel structures 620 extend axially and have a length L2 that is less than a length L of the substrate 610 in the illustrated example. In other examples, one or more of the hydrogel structures 620 can have a length L2 that is the same as the length L of the substrate. Each of the hydrogel structures 620 may have the same length L2, or the lengths L2 may vary. When the substrate 610 is an annular form placed around the frame of a prosthetic heart valve, the hydrogel structures 620 may be spaced around the entire annular substrate.

FIG. 7 shows a sealing member 700 comprising a substrate 710 and a plurality of hydrogel structures 720 extending across a width W of the substrate 710 (the width W being in the circumferential direction). Each of the hydrogel structures 720 has a width W2 that is less than the width W. Each of the hydrogel structures 620 may have the same with W2, or the widths W2 may vary. When the substrate 610 is an annular form around the frame of a prosthetic heart valve, the hydrogel structures 620 extend circumferentially around the substrate 610.

In any of the examples of FIGS. 5-7, the hydrogel structures 520, 620, 720 can be discrete hydrogel structures formed on a substrate, or they can be hydrogel structures formed in (e.g., laser cut or etched) a continuous layer of hydrogel material formed on the substrate. Moreover, in other examples, a sealing member can comprise a substrate and any combination of hydrogel structures 120, 222, 520, 620, 720.

In any of the embodiments shown in FIGS. 5-7, top width W_{T}, taper angle α, and pitch p, of the hydrogel structures are as previously discussed with respect to FIG. 3. In any of the embodiments shown in FIGS. 5-7, the non-expanded thickness T1 and expanded thickness T2 are as previously described with respect to FIG. 3. In any of the embodiments shown in FIGS. 5-7, when a hydrogel layer is disposed on the substrate and the hydrogel layer has regions of a first average thickness defining the hydrogel structures alternating with regions of a second average thickness, the first and second average thicknesses T3 and T4 may be as previously described with respect to FIG. 4.

Embodiments of the disclosed smart, multifunctional sealing member comprise a substrate comprising a substrate material. The substrate material may be any material suitable for an implantable prosthesis. In some embodiments, the substrate material is a textile, such as a textile formed from polymer yarns or fibers. Suitable polymers include, but are not limited to, polyethylene terephthalate (PET) or ultra-high molecular weight (UHMW) polyethylene. In some examples, the substrate material is a woven, braided, or knitted textile or fabric, such as woven, braided, or knitted PET, or alternatively, a non-woven textile, such as a felt (e.g., a PET or UHMW felt). In other examples, the substrate material is a plush textile, such as a velour. In other embodiments, the substrate can be natural tissue, such as bovine or porcine pericardium (or pericardium from other sources).

In any of the foregoing or following embodiments, the hydrogel structures 120, 224 may comprise a natural hydrogel, a synthetic hydrogel, or a combination of a natural hydrogel and a synthetic hydrogel. Naturally occurring hydrogels include, but are not limited to, polysaccharides, collagen, peptides, cyclodextrin, and chitosan (some polysaccharides, peptides, and cyclodextrins may be synthetic). Other exemplary hydrogels, including synthetic hydrogels are discussed in detail below. The hydrogel may be biosorbable or biostable. In some embodiments, the hydrogel is thermoplastic or thermoset. The hydrogel may be hydrophilic or hydrophobic in nature.

In any of the foregoing or following embodiments, the hydrogel may improve and accelerate healthy endothelialization and/or reduce foreign body reaction when a prosthesis comprising the sealing member is implanted into a subject. For example, the hydrogel ring may improve endothelial cell adhesion, migration, and/or proliferation. Endothelialization may reduce hemolysis, reduce thrombosis/promote anticoagulation, reduce inflammation, or any combination thereof.

The hydrogel is stimulus responsive. The hydrogel may respond to one or more stimuli including, but not limited to, exposure to fluids (e.g., water, saline, buffers, or bodily fluids, such as blood), a temperature change, a pH change, irradiation with light, exposure to ultrasound, application of an electric field, application of a magnetic field, or any combination thereof. The hydrogel structure undergoes a stimulus-induced change in volume, stiffness (e.g., measured by durometer hardness and/or Young's modulus), hydrophobicity, or any combination thereof.

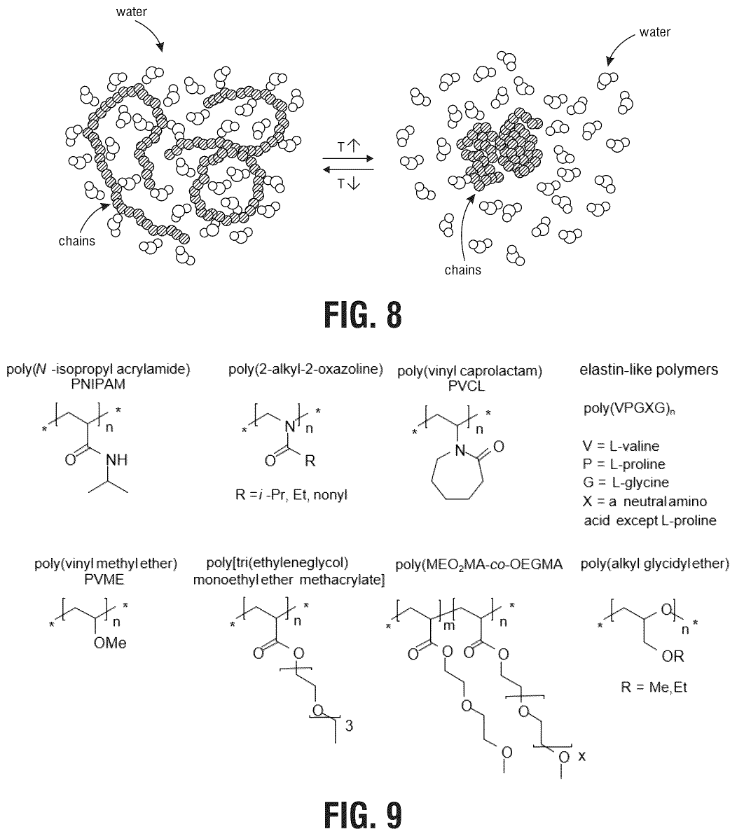

In one embodiment, exposure to fluids (such as a fluid with a decreased ionic strength or a fluid with a different chemical composition), and/or an increase in temperature causes the hydrogel to swell as the fluid increases space between the polymer chains. In another embodiment, an increase in temperature may increase stiffness of the hydrogel. FIG. 8 illustrates an exemplary embodiment in which the polymer molecules reversibly transition between an expanded coil conformation and a compact globular conformation when temperature is increased or decreased, resulting in increased stiffness at higher temperatures. In another embodiment, a light-responsive hydrogel may undergo conformational changes in the polymer molecules, such as photoisomerization, photodimerization, when exposed to light, resulting in volume changes as fluids are taken up or released. Alternatively, light irradiation may induce irreversible or reversible changes by cleaving the polymer backbone and/or crosslinks between polymer chains. For instance, some hydrogels may include reversible covalent bonds that are cleaved upon UV irradiation, but spontaneously reform when the light is removed. In another embodiment, the hydrogel may be responsive to an electric or magnetic field, and may reversibly or irreversibly swell or shrink when the stimulus is applied and removed. In still another embodiment, the hydrogel may be responsive to pH changes, which may induce ionization of functional groups, enabling charge-density redistribution and swelling as groups on adjacent polymer molecules with similar charges electrostatically repel one another and fluid diffuses into the spaces between the polymer molecules; the hydrogel molecules may include anionic groups which ionize at high pH and/or cationic groups which ionize at low pH. In yet another embodiment, ultrasound stimulation may reversibly or irreversibly disrupt crosslinking between polymer chains, such as disrupting noncovalent crosslinking

In any of the foregoing or following embodiments, the hydrogel structure 120, 224 may be in a flattened, unobtrusive state with the hydrogel structure having a non-expanded thickness T1 in an ex vivo environment. The flattened conformation facilitates delivery of a prosthesis (e.g., a prosthetic heart valve) comprising the sealing member into a desired site in a subject's body by reducing the overall crimp profile of the prosthesis. After being introduced into the patient's body, such as at or near a desired implantation site or after being deployed at the implantation site, the hydrogel structure 120, 224 is subjected to one or more stimuli, resulting in expansion of the hydrogel structure to its expanded thickness T2 as fluid is absorbed by the hydrogel. The stimulus also may increase stiffness of the hydrogel structure 120, 224 by inducing a conformational change in the polymer chains as the expanded coil conformation at room temperature (e.g., 25° C.) collapses into compact globuli at body temperature (e.g., 37° C.). When the sealing member comprises a plurality of the hydrogel structures 120, 224, expansion creates a labyrinth seal, as shown in FIGS. 3 and 4.

In any of the foregoing or following embodiments, the natural or synthetic hydrogel may comprise a poloxamer, a poly(N-alkylacrylamide), a poly(n-vinylcaprolactam), a poly(alkyloxazoline), a poly(vinyl alkyl ether), a poly(alkyl glycidyl ether), a poly(methacrylic acid), a poly(alkylmethacrylate), a poly(acrylic acid), a poly(vinylpyridine), a poly(vinylimidazole), a poly(thiophene), a poly(alkyloxazoline),a polyamine, a sulfonated polystyrene, ethylene-vinyl acetate, polyurethane, poly(ethylene oxide) (PEO), poly(propylene oxide) (PPO), a polyketal, a polyacetal, a polylactide, a polyglycolide, a polysaccharide, collagen, a peptide, cyclodextrin, a nitrocatechol-terminated polymer, or any combination or copolymer thereof.

In one implementation, the hydrogel is temperature responsive and comprises a poloxamer, a poly(N-alkylacrylamide), a poly(n-vinylcaprolactam), a poly(alkyloxazoline), a poly(vinyl alkyl ether), a poly(alkyl glycidyl ether), a polyurethane, cellulose, xyloglucan, chitosan, elastin and derivatives thereof, poly(methoxydiethylene glycol methacrylate), poly(methoxytriethylene glycol methacrylate), or any combination thereof. In some embodiments, the temperature-responsive hydrogel may comprise a combination of a polymer and one or more lipids or sterols. Chemical structures of exemplary thermoresponsive polymers are shown in FIG. 9. In another implementation, the hydrogel is pH responsive and comprises a poly(methacrylic acid), a poly(acrylic acid), a poly(vinylpyridine), a poly(vinylimidazole), a polyurethane, or any combination thereof. In yet another implementation, the hydrogel is light responsive and comprises a poly(acrylamide), a polyketal, a polyacetal, a nitrocatechol-terminated polymer, or any combination thereof. In some examples, polyketals and polyacetals are photolyzed by UV light (e.g., at 248 nm) into carbonyl and hydroxyl products through zwitterionic intermediates. In still another implementation, the hydrogel is responsive to electric fields and comprises a sulfonated polystyrene, a poly(thiophene), poly(ethyloxazoline), or any combination thereof. In another implementation, the hydrogel is ultrasound responsive and comprises a biodegradable polymer (e.g., polylactide or polyglycolide), or a non-biodegradable polymer (e.g., ethylene-vinyl acetate, a poly(lactide-co-glycolide), or a combination thereof.

In one example, polyethylene oxide/polypropylene oxide co-block or triblock polymers are temperature and ultrasound responsive. In another example, polymethacrylates are pH and ultrasound responsive. In yet another example, poly(N-isopropylacrylamide) (PNIPAM) is thermoresponsive with a sharp lower critical solution temperature (LCST)-type transition at around 33° C. In still another example, the 7-membered ring, poly(N-vinylcaprolactam) (PVCap) polymers have a cloud point (CP) in water that is close to body temperature at 34° C. to 37° C.

Exemplary hydrogels include, but are not limited to:
a PEO-PPO co-block polymer;
a PEO-PPO-PEO triblock polymer;
poly(ethyleneglycol)-b-(2-(dimethylamino)ethyl methacrylate-co-methyl methacrylate) (PEG-b-(DMAEM-co-MMA);
poly(methoxydiethylene glycol methacrylate (PmDEGMA);
poly(methoxytriethylene glycol methacrylate (PmTEGMA);
poly(N-isopropylacrylamide) (PNIPAM);
PNIPAM-NH₂;
poly(NIPAM-co-acrylic acid) (P(NIPAM-co-AA));
poly(NIPAM-acrylamide-allylamine);
poly(NIPAM-co-N,N-dimethylacrylamide (P(NIPAM-co-N,NDMAM));
cholesterol-graft-poly[NIPAM-co-N-(hydroxymethyl) acrylamide] (CHOL-g-PNIPAM-co-NHMAAM);
poly(NIPAM-co-N,NDMAM)-b-poly(D,L-lactide-co-glycolide) (P(NIPAM-co-N,NDMAM-b-PLGA);
poly(NIPAM-co-N,NDMAM)-b-poly(lactide) (P(NIPAM-co-N,NDMAM-b-PLa);
poly(NIPAM-co-acrylamide) (P(NIPAM-co-AAM));
poly(NIPAM-co-N,NDMAM)-b-poly(lactide-co-caprolactone) (P(NIPAM-co-N,NDMAM-b-P(LA-co-CL);
poly[NIPAM-b-poly(butyl methacrylate)] (P(NIPAM-b-PBMA);
Fe₃O₄—P(NIPAM);
poly[monomethyl oligo (ethylene glycol)acrylate-2-(5,5-dimethyl-1,3-dioxan-2-yloxy) ethyl acrylate] (P(MOEGA-DMDEA);
poly(N-vinylcaprolactam);
a thermoplastic polyurethane;
poly(-isopropoyl-2-oxazoline);
poly(-ethyl-2-oxazoline), poly(-nonyl-2-oxazoline);
poly(L-valine-L-proline-L-glycine-X-L-glycine)ₙ where X is a neutral amino acid other than L-proline;
poly(vinyl methyl ether);
poly[tri(ethylene glycol) monoethyl ether methacrylate];
poly[2-(2-methoxyethoxy) ethyl methacrylate-co-oligo (ethylene glycol) methacrylate];
poly(methyl glycidyl ether);
poly(ethyl glycidyl ether);
poly(ethylene glycol)-coated hollow gold nanospheres (PEG-HAuNS);
a polysaccharide;
collagen;
cyclodextrin;
chitosan;
chitosan-graft-polyethylenimine;
elastin;
1,2-diplamitoyl-sn-glycero-3-phosphatidylcholine:dipalmitoyl phospatidylglycerol:1-stearoyl-2-hydroxy-sn-glycero-3-phosphocholine:poly(ethylene glycol) methyl ether (Mₙ 2000):1,2,-distearoyl-sn-glycero3-phosphoethanolamine (DPPC:DPPG:MSPC:mPEG2000-DSPE);
DPPC:MSPC:DSPE-PEG2000;
DPPC:1,2-distearoyl-sn-glycero-phosphocholine:1,2-dipalmitoyl-3-trimethylammonium-propane:DSPE-polyethylene glycol (Mₙ2000) (DPPC:DSPC:DPTAP:DSPE:PEG2000);
DPPC:monopalmitoyl phosphatidylcholine:dipalmitoyl-sn-glycero-3-phosphatidylethanolamine:PEG2000 (DPPC-MPPC-DPPE-PEG2000);
DPPC:hydrogenated soy phosphatidylcholine-CHOL:DPPE:PEG2000 (DPPC:HSPC:CHOL:DPPE:PEG2000);

DPPC:CHOL:DSPE:PEG2000:DSPE:PEG2000-folate;
and combinations thereof.

Table 1 provides a non-exhaustive list of exemplary hydrogels that are thermoresponsive in a temperature range that encompasses human body temperature (37° C.), and exhibit a change in stiffness as the polymer molecules transition between an expanded coil conformation and a compact, globular conformation (e.g., as shown in FIG. 8).

TABLE 1

| | Switching |
|---|---|
| | temperature |
| Polymer Chemistry | (° C.) |
| P(NIPAM-co-AA) | 5-50 |
| P(NIPAM) | 33-38 |
| PEG-HAuNS | 40 |
| P(NIPAM-acrylamide-allylamine) | 40 |
| Chitosan-g-PEI | 28 |
| Elastin-like | 27 |
| P(NIPAM-co-N,NDMAM) | 37-40 |
| CHOL-g-P[NIPAM-co-N-(hydroxymethyl) acrylamide] | 35-40 |
| P(NIPAM-co-N,NDMAM)-b-PLGA | 39-40 |
| P(NIMAM-co-N,NDMAM)-b-PLa | 39-41 |
| P(NIPAM-co-AAm) | 39-42 |
| P(MOEGA-DMDEA) | 24-35 |
| P(NIPAM-co-N,NDMAM)-b-P(LA-co-CL) | 40 |
| P(NIPAM-b-PBMA) | 32-33 |
| *Pluronic ® F127 (a PEG-PPG-PEG block copolymer) | 37 |
| (Fe₃O₄)—P(NIPAM) | 35 |
| PEGylated liposomes | 40-43 |
| DPPC:DPPG:MSPC:mPEG2000-DSPE | 37-42 |
| DPPC/MSPC/DSPE-PEG2000 | 32 |
| P(NIPAM)-NH₂(amino-terminated) | 20 |
| DPPC:DSPC:DPTAP:DSPE:PEG2000 | 39-40 |
| DPPC:MPPC:DPPE-PEG2000, | 39-40 |
| DPPC:HSPC:CHOL:DPPE-PEG2000 | 38-40 |
| DPPC:CHOL:DSPE-PEG2000:DSPE-PEG2000-folate | 42 |

*available from Sigma-Aldrich Corp., St. Louis, MO

### III. Method of Making a Smart Multifunctional Sealing Member

A method of making a smart multifunctional sealing member as disclosed herein includes providing a substrate comprising a substrate material, the substrate having an outwardly facing surface and an inwardly facing surface, the substrate having a length L; and forming an hydrogel structure on the outwardly facing surface of the substrate, the hydrogel structure comprising a hydrogel. In some embodiments, a plurality of hydrogel structures is formed.

The hydrogel structure, or structures, may be formed by any of several suitable methods. In one implementation, the hydrogel is extruded or molded onto the outwardly facing surface of the substrate (e.g., a woven, knitted, or braided fabric) to form one or more hydrogel structures that are thermally attached to the outwardly facing surface. In another implementation, a hydrogel structure, or structures, is mechanically attached to the outwardly facing surface of the substrate. In yet another implementation, a hydrogel structure, or structures, is chemically attached to the outwardly facing surface of the substrate. In still another implementation, a hydrogel layer is applied to the outwardly facing surface of the substrate by dip coating or spray coating, and the hydrogel layer is molded or etched to form the hydrogel structure or structures. Dip coating or spray coating may be combined with thermal treatment, mechanical attachment, or chemical attachment to attach the hydrogel layer to the outwardly facing surface of the substrate.

In some embodiments, the hydrogel structure is mechanically attached to the outwardly facing surface of the annular substrate. The hydrogel structure may be mechanically attached using pressure or ultrasound to modify the surface chemistry or morphology of the annular substrate.

In some embodiments, the hydrogel structure is chemically attached to the outwardly facing surface of the substrate. Chemical attachment may be performed by any suitable technique. In one example, functional groups of the hydrogel may be crosslinked to functional groups of the substrate material using ultraviolet irradiation. In another example, the hydrogel structure may be applied to the outwardly facing surface of the substrate, followed by hydrolyzing or oxidizing functional groups of the hydrogel, functional groups of the substrate material, or functional groups of the hydrogel and the substrate material, whereby functional groups of the hydrogel react with functional groups of the substrate material. Hydrolysis may be performed with acids or bases, such as acetic acid or sodium hydroxide. Oxidation may be performed with any suitable oxidizing agent, such as hydrogen peroxide.

In some embodiments, e.g., as shown in FIG. 4, a layer of the hydrogel is dip coated or spray coated onto the outwardly facing surface of the substrate, and the hydrogel layer is subsequently molded or etched to form the hydrogel structure. In certain embodiments, with reference to FIG. 4, a laser is used to etch the hydrogel layer to provide a first region 222 having a first average thickness T1 that defines the hydrogel structure and adjacent regions 224 having a second average thickness T2, wherein the second average thickness T2 is less than the first average thickness T1. In some examples, the hydrogel layer is etched to provide a plurality of first regions 222 alternating with regions 224.

In some embodiments, the hydrogel structure is a linear structure extending across the width of the substrate, or a plurality of spaced apart linear structures extending across the width of the substrate (e.g., as shown in FIG. 2). In some implementations, the linear structure may have a zigzag configuration (e.g., as shown in FIG. 5). When the substrate is an annular substrate, the linear structure, or structures, extends circumferentially around the annular substrate.

In some embodiments, the hydrogel structure is a plurality of spaced apart linear structures extending along the length of the substrate, each linear structure having a length L2 equal or less than the length L of the substrate. When the substrate is an annular substrate, the linear structures extend axially.

In some embodiments, the hydrogel structure is a plurality of spaced-apart discontinuous linear structures, each linear structure extending across a portion of a width of the substrate (e.g., as shown in FIG. 7). When the substrate is an annular substrate, each of the linear structures extends circumferentially around a portion of the annular substrate. Each of the discontinuous linear structures may be spaced apart both laterally and vertically from adjacent structures.

In any of the foregoing embodiments, the sealing member may be formed in a flattened or unrolled configuration. Once formed, the sealing member can be assembled on the frame (e.g., frame 12) of a prosthetic heart valve, such as by wrapping the sealing member around the outer surface of the frame and attaching the sealing member to the frame, such as with sutures, to form an annular outer skirt of the prosthetic heart valve.

In other embodiments, the sealing member can be mounted along an inner surface of the frame of a prosthetic heart valve to form an annular inner skirt of the prosthetic heart valve. For example, the inner skirt 16 of the prosthetic heart valve 10 can be replaced with a sealing member having one or more hydrogel structures made in accordance with any disclosed embodiment. In such embodiments, the hydrogel structures can be positioned to protrude outwardly through the cells (openings) of the frame and contact the surrounding anatomy, in which case an outer skirt need not be included.

### IV. Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame configured to be radially compressible and expandable between a radially compressed state and a radially expanded state; a valvular structure disposed within the annular frame and configured to regulate flow of blood through the annular frame in one direction; and a sealing member comprising a fabric substrate and a hydrogel structure attached to the fabric substrate, the hydrogel structure comprising a stimulus-responsive hydrogel and having an exposed outer surface configured to seal against tissue surrounding the prosthetic heart valve when implanted in a patient's body.

Example 2. The prosthetic heart valve of any example herein, particularly example 1, wherein the sealing member comprises an outer skirt that extends around an outer surface of the annular frame.

Example 3. The prosthetic heart valve of any example herein, particularly example 1, wherein the sealing member comprises an inner skirt that extends along an inner surface of the annular frame.

Example 4. The prosthetic heart valve of any example herein, particularly any one of examples 1-3, wherein the sealing member is stitched to struts of the annular frame.

Example 5. The prosthetic heart valve of any example herein, particularly any one of examples 1-4, wherein the stimulus-responsive hydrogel undergoes a change in volume, stiffness, or both induced by a stimulus, the stimulus comprising a temperature change, a pH change, an ionic strength change, a solvent composition change, application of an electric field, application of a magnetic field, application of ultrasound, exposure to light, or any combination thereof.

Example 6. The prosthetic heart valve of any example herein, particularly any one of examples 1-5, wherein the hydrogel structure is bonded to the fabric substrate.

Example 7. The prosthetic heart valve of any example herein, particularly any one of examples 1-6, wherein the fabric substrate comprises polyethylene terephthalate or polyethylene.

Example 8. The prosthetic heart valve of any example herein, particularly any one of examples 1-7, wherein the fabric substrate comprises a woven, braided, or knitted fabric.

Example 9. The prosthetic heart valve of any example herein, particularly any one of examples 1-8, wherein the stimulus-responsive hydrogel comprises a natural or synthetic hydrogel.

Example 10. The prosthetic heart valve of any example herein, particularly example 9, wherein the natural or synthetic hydrogel comprises a poloxamer, a poly(N-alkylacrylamide), a poly(n-vinylcaprolactam), a poly(alkyloxazoline), a poly(vinyl alkyl ether), a poly(alkyl glycidyl ether), a poly(methacrylic acid), a poly(alkylmethacrylate), a poly(acrylic acid), a poly(vinylpyridine), a poly(vinylimidazole), a poly(thiophene), a poly(alkyloxazoline),a polyamine, a sulfonated polystyrene, ethylene-vinyl acetate, polyurethane, poly(ethylene oxide) (PEO), poly(propylene oxide) (PPO), a polyketal, a polyacetal, a polylactide, a polyglycolide, a polysaccharide, collagen, a peptide, cyclodextrin, a nitrocatechol-terminated polymer, or any combination or copolymer thereof.

Example 11. The prosthetic heart valve of any example herein, particularly example 9, wherein the natural or synthetic hydrogel comprises a PEO-PPO co-block polymer; a PEO-PPO-PEO triblock polymer; poly(ethyleneglycol)-b-(2-(dimethylamino)ethyl methacrylate-co-methyl methacrylate) (PEG-b-(DMAEM-co-MMA); poly(methoxydiethylene glycol methacrylate (PmDEGMA); poly(methoxytriethylene glycol methacrylate (PmTEGMA); poly(N-isopropylacrylamide) (PNIPAM); PNIPAM-NH₂; poly(NIPAM-co-acrylic acid) (P(NIPAM-co-AA)); poly(NIPAM-acrylamide-allylamine); poly(NIPAM-co-N,N-dimethylacrylamide (P(NIPAM-co-N,NDMAM)); cholesterol-graft-poly[NIPAM-co-N-(hydroxymethyl) acrylamide] (CHOL-g-PNIPAM-co-NHMAAM); poly(NIPAM-co-N,NDMAM)-b-poly(D,L-lactide-co-glycolide) (P(NIPAM-co-N,NDMAM-b-PLGA); poly(NIPAM-co-N,NDMAM)-b-poly(lactide) (P(NIPAM-co-N,NDMAM-b-PLa); poly(NIPAM-co-acrylamide) (P(NIPAM-co-AAM)); poly(NIPAM-co-N,NDMAM)-b-poly(lactide-co-caprolactone) (P(NIPAM-co-N,NDMAM-b-P(LA-co-CL); poly[NIPAM-b-poly(butyl methacrylate)] (P(NIPAM-b-PBMA); Fe₃O₄-P(NIPAM); poly[monomethyl oligo (ethylene glycol)acrylate-2-(5,5-dimethyl-1,3-dioxan-2-yloxy) ethyl acrylate] (P(MOEGA-DMDEA); poly(N-vinylcaprolactam); a thermoplastic polyurethane; poly(2-isopropoyl-2-oxazoline); poly(2-ethyl-2-oxazoline), poly(-nonyl-2-oxazoline); poly(L-valine-L-proline-L-glycine-X-L-glycine)ₙ where X is a neutral amino acid other than L-proline; poly(vinyl methyl ether); poly[tri(ethylene glycol) monoethyl ether methacrylate]; poly[2-(2-methoxyethoxy) ethyl methacrylate-co-oligo (ethylene glycol) methacrylate]; poly(methyl glycidyl ether); poly(ethyl glycidyl ether); poly(ethylene glycol)-coated hollow gold nanospheres (PEG-HAuNS); a polysaccharide; collagen; cyclodextrin; chitosan; chitosan-graft-polyethylenimine; elastin; 1,2-diplamitoyl-sn-glycero-3-phosphatidylcholine:dipalmitoyl phospatidylglycerol:1-stearoyl-2-hydroxy-sn-glycero-3-phosphocholine:poly(ethylene glycol) methyl ether (Mₙ2000):1,2,-distearoyl-sn-glycero3-phosphoethanolamine (DPPC:DPPG:MSPC:mPEG2000-DSPE); DPPC:MSPC:DSPE-PEG2000; DPPC:1,2-distearoyl-sn-glycero-phosphocholine:1,2-dipalmitoyl-3-trimethylammonium-propane: DSPE-polyethylene glycol (Mₙ2000) (DPPC:DSPC:DPTAP:DSPE:PEG2000); DPPC:monopalmitoyl phosphatidylcholine:dipalmitoyl-sn-glycero-3-phosphatidylethanolamine:PEG2000 (DPPC-MPPC-DPPE-PEG2000); DPPC:hydrogenated soy phosphatidylcholine-CHOL:DPPE:PEG2000 (DPPC:HSPC:CHOL:DPPE:PEG2000); DPPC:CHOL:DSPE:PEG2000:DSPE:PEG2000-folate; or any combination thereof.

Example 12. A sealing member for an implantable prosthesis, the sealing member comprising: a substrate comprising a substrate material, the substrate having an outwardly facing surface and an inwardly facing surface; and a stimulus-responsive hydrogel disposed on the outwardly facing surface of the substrate.

Example 13. The sealing member of any example herein, particularly example 12, wherein the stimulus-responsive hydrogel undergoes a change in volume, stiffness, or both induced by a stimulus, the stimulus comprising a temperature change, a pH change, an ionic strength change, a solvent composition change, application of an electric field, application of a magnetic field, application of ultrasound, exposure to light, or any combination thereof.

Example 14. The sealing member of any example herein, particularly example 12 or example 13, wherein the stimulus-responsive hydrogel, in an ex vivo environment, has a first non-expanded thickness T1 measured from the outwardly facing surface of the substrate to an outer surface of the stimulus-responsive hydrogel.

Example 15. The sealing member of any example herein, particularly example 14, wherein the stimulus-responsive hydrogel, in an in vivo environment, has a second expanded thickness T2, wherein T2 is greater than T1.

Example 16. The sealing member of any example herein, particularly any one of examples 12-15, wherein the substrate material comprises polyethylene terephthalate or polyethylene.

Example 17. The sealing member of any example herein, particularly any one of examples 12-16, wherein the substrate comprises a woven, braided, or knitted fabric.

Example 18. The sealing member of any example herein, particularly any one of examples 12-17, wherein the stimulus-responsive hydrogel comprises a natural or synthetic hydrogel.

Example 19. The sealing member of any example herein, particularly example 18, wherein the natural or synthetic hydrogel comprises a poloxamer, a poly(N-alkylacrylamide), a poly(n-vinylcaprolactam), a poly(alkyloxazoline), a poly(vinyl alkyl ether), a poly(alkyl glycidyl ether), a poly(methacrylic acid), a poly(alkylmethacrylate), a poly(acrylic acid), a poly(vinylpyridine), a poly(vinylimidazole), a poly(thiophene), a poly(alkyloxazoline),a polyamine, a sulfonated polystyrene, ethylene-vinyl acetate, polyurethane, poly(ethylene oxide) (PEO), poly(propylene oxide) (PPO), a polyketal, a polyacetal, a polylactide, a polyglycolide, a polysaccharide, collagen, a peptide, cyclodextrin, a nitrocatechol-terminated polymer, or any combination or copolymer thereof.

Example 20. The sealing member of any example herein, particularly example 18, wherein the natural or synthetic hydrogel comprises a PEO-PPO co-block polymer; a PEO-PPO-PEO triblock polymer; poly(ethyleneglycol)-b-(2-(dimethylamino)ethyl methacrylate-co-methyl methacrylate) (PEG-b-(DMAEM-co-MMA); poly(methoxydiethylene glycol methacrylate (PmDEGMA); poly(methoxytriethylene glycol methacrylate (PmTEGMA); poly(N-isopropylacrylamide) (PNIPAM); PNIPAM-NH₂; poly(NIPAM-co-acrylic acid) (P(NIPAM-co-AA)); poly(NIPAM-acrylamide-allylamine); poly(NIPAM-co-N,N-dimethylacrylamide (P(NIPAM-co-N,NDMAM)); cholesterol-graft-poly[NIPAM-co-N-(hydroxymethyl) acrylamide] (CHOL-g-PNIPAM-co-NHMAAM); poly(NIPAM-co-N,NDMAM)-b-poly (D,L-lactide-co-glycolide) (P(NIPAM-co-N,NDMAM-b-PLGA); poly(NIPAM-co-N,NDMAM)-b-poly(lactide) (P(NIPAM-co-N,NDMAM-b-PLa); poly(NIPAM-co-acrylamide) (P(NIPAM-co-AAM)); poly(NIPAM-co-N,NDMAM)-b-poly (lactide-co-caprolactone) (P(NIPAM-co-N,NDMAM-b-P(LA-co-CL); poly[NIPAM-b-poly (butyl methacrylate)] (P(NIPAM-b-PBMA); Fe₃O₄-P(NIPAM); poly[monomethyl oligo (ethylene glycol)acrylate-2-(5,5-dimethyl-1,3-dioxan-2-yloxy) ethyl acrylate] (P(MOEGA-DMDEA); poly(N-vinylcaprolactam); a thermoplastic polyurethane; poly(2-isopropoyl-2-oxazoline); poly(2-ethyl-2-oxazoline), poly(2-nonyl-2-oxazoline); poly(L-valine-L-proline-L-glycine-X-L-glycine)ₙ where X is a neutral amino acid other than L-proline; poly(vinyl methyl ether); poly[tri(ethylene glycol) monoethyl ether methacrylate]; poly[2-(2-methoxyethoxy) ethyl methacrylate-co-oligo (ethylene glycol) methacrylate]; poly(methyl glycidyl ether); poly(ethyl glycidyl ether); poly(ethylene glycol)-coated hollow gold nanospheres (PEG-HAuNS); a polysaccharide; collagen; cyclodextrin; chitosan; chitosan-graft-polyethylenimine; elastin; 1,2-diplamitoyl-sn-glycero-3-phosphatidylcholine:dipalmitoyl phospatidylglycerol:1-stearoyl-2-hydroxy-sn-glycero-3-phosphocholine:poly (ethylene glycol) methyl ether (Mₙ2000):1,2,-distearoyl-sn-glycero3-phosphoethanolamine (DPPC:DPPG:MSPC:mPEG2000-DSPE); DPPC:MSPC:DSPE-PEG2000; DPPC:1,2-distearoyl-sn-glycero-phosphocholine:1,2-dipalmitoyl-3-trimethylammonium-propane: DSPE-polyethylene glycol (Mₙ2000) (DPPC:DSPC:DPTAP:DSPE:PEG2000); DPPC:monopalmitoyl phosphatidylcholine:dipalmitoyl-sn-glycero-3-phosphatidylethanolamine:PEG2000 (DPPC-MPPC-DPPE-PEG2000); DPPC:hydrogenated soy phosphatidylcholine-CHOL:DPPE:PEG2000 (DPPC:HSPC:CHOL:DPPE:PEG2000); DPPC:CHOL:DSPE:PEG2000:DSPE:PEG2000-folate; or any combination thereof.

Example 21. The sealing member of any example herein, particularly any one of examples 12-20, in combination with an implantable prosthesis, wherein the implantable prosthesis is a prosthetic heart valve comprising an annular frame and the sealing member is mounted on an inner surface or an outer surface of the annular frame.

Example 22. A sealing member for an implantable prosthesis comprising an outer skirt, the outer skirt comprising: a substrate comprising a substrate material, the substrate having an outwardly facing surface and an inwardly facing surface, the substrate having a length L; and a hydrogel structure disposed on the outwardly facing surface of the substrate, the hydrogel structure comprising a stimulus-responsive hydrogel.

Example 23. The sealing member of any example herein, particularly example 22, wherein the stimulus-responsive hydrogel undergoes a change in volume, stiffness, or both induced by a stimulus, the stimulus comprising a temperature change, a pH change, an ionic strength change, a solvent composition change, application of an electric field, application of a magnetic field, application of ultrasound, exposure to light, or any combination thereof.

Example 24. The sealing member of any example herein, particularly example 22 or example 23, wherein the hydrogel structure, in an ex vivo environment, has a first non-expanded thickness T1 measured from the outwardly facing surface of the substrate to an outer surface of the hydrogel structure.

Example 25. The sealing member of any example herein, particularly example 24, wherein the hydrogel structure, in an in vivo environment, has a second expanded thickness T2, wherein T2 is greater than T1.

Example 26. The sealing member of any example herein, particularly any one of examples 22-25, wherein a top width W_{T} of the hydrogel structure at the outer surface, in the in vivo environment, is 0.2 mm to 0.4 mm

Example 27. The sealing member of any example herein, particularly example 26, wherein the second expanded thickness T2 is from 10×W_{T} to 12×W_{T}.

Example 28. The sealing member of any example herein, particularly any one of examples 22-27, further comprising a plurality of spaced-apart hydrogel structures disposed on the outwardly facing surface of the substrate.

Example 29. The sealing member of any example herein, particularly any one of examples 22-28, wherein the hydrogel structure is an annular hydrogel ring.

Example 30. The sealing member of any example herein, particularly example 29, wherein the annular hydrogel ring has a base width W_{B} that is less than the length L of the substrate.

Example 31. The sealing member of any example herein, particularly example 30, wherein the base width W_{B} of the hydrogel structure is greater than or equal to a top width W_{T} of the hydrogel structure at an outer surface of the hydrogel structure.

Example 32. The sealing member of any example herein, particularly example 31, wherein the hydrogel structure tapers from the base width W_{B} to the top width W_{T} at an angle α of 0 degrees to 20 degrees.

Example 33. The sealing member of any example herein, particularly any one of examples 29-32, wherein the annular hydrogel ring has a zigzag configuration.

Example 34. The sealing member of any example herein, particularly any one of examples 29-33, wherein the hydrogel structure comprises a plurality of annular hydrogel rings.

Example 35. The sealing member of any example herein, particularly example 34, wherein a pitch p of the plurality of annular hydrogel rings is from 10×W_{T} to 18×W_{T}.

Example 36. The sealing member of any example herein, particularly example 28, wherein the plurality of spaced-apart hydrogel structures comprises a plurality of spaced-apart axially extending hydrogel structures having a length L2 less than the length L of the substrate.

Example 37. The sealing member of any example herein, particularly example 36, wherein each of the plurality of spaced-apart axially extending hydrogel structures has a base width W_{B} that is greater than or equal to a top width W_{T} at an outer surface of the hydrogel structure.

Example 38. The sealing member of any example herein, particularly example 37, wherein each of the plurality of spaced-apart axially extending hydrogel structures tapers from the base width W_{B} to the top width W_{T} at an angle a of 0 degrees to 20 degrees.

Example 39. The sealing member of any example herein, particularly example 28, wherein the plurality of spaced-apart hydrogel structures comprises a plurality of discontinuous circumferential structures, each hydrogel structure extending across a portion of the substrate.

Example 40. The sealing member of any example herein, particularly example 39, wherein each hydrogel structure has a base width W_{B} that is less than the length L of the substrate.

Example 41. The sealing member of any example herein, particularly example 40, wherein the base width W_{B} is greater than or equal to a top width W_{T} of the hydrogel structure at an outer surface of the hydrogel structure.

Example 42. The sealing member of any example herein, particularly example 41, wherein each hydrogel structure tapers from the base width W_{B} to the top width W_{T} at an angle α of 0 degrees to 20 degrees.

Example 43. The sealing member of any example herein, particularly any one of examples 22-27, wherein the hydrogel structure is a layer of the stimulus-responsive hydrogel disposed on the outwardly facing surface of the substrate.

Example 44. The sealing member of any example herein, particularly example 43, wherein the layer of the stimulus-responsive hydrogel comprises regions of a first average thickness T3 defining a plurality of spaced-apart hydrogel structures alternating with regions of a second average thickness T4, wherein the first average thickness T3 is greater than the second average thickness T4.

Example 45. The sealing member of any example herein, particularly example 44, wherein the plurality of spaced-apart hydrogel structures is a plurality of spaced-apart annular hydrogel rings.

Example 46. The sealing member of any example herein, particularly example 45, wherein each of the plurality of spaced-apart annular hydrogel rings has a zigzag configuration.

Example 47. The sealing member of any example herein, particularly example 44, wherein the plurality of spaced-apart hydrogel structures comprises a plurality of spaced-apart axially extending hydrogel structures having a length L2 less than the length L of the substrate.

Example 48. The sealing member of any example herein, particularly example 44, wherein the plurality of spaced-apart hydrogel structures comprises a plurality of discontinuous circumferential structures, each hydrogel structure extending around a portion of the substrate.

Example 49. The sealing member of any example herein, particularly any one of examples 22-48, wherein the stimulus-responsive hydrogel comprises a natural or synthetic hydrogel.

Example 50. The sealing member of any example herein, particularly example 49, wherein the natural or synthetic hydrogel comprises a poloxamer, a poly(N-alkylacrylamide), a poly(n-vinylcaprolactam), a poly(alkyloxazoline), a poly(vinyl alkyl ether), a poly(alkyl glycidyl ether), a poly(methacrylic acid), a poly(alkylmethacrylate), a poly(acrylic acid), a poly(vinylpyridine), a poly(vinylimidazole), a poly(thiophene), a poly(alkyloxazoline),a polyamine, a sulfonated polystyrene, ethylene-vinyl acetate, polyurethane, poly(ethylene oxide) (PEO), poly(propylene oxide) (PPO), a polyketal, a polyacetal, a polylactide, a polyglycolide, a polysaccharide, collagen, a peptide, cyclodextrin, a nitrocatechol-terminated polymer, or any combination or copolymer thereof.

Example 51. The sealing member of any example herein, particularly example 49, wherein the natural or synthetic hydrogel comprises a PEO-PPO co-block polymer; a PEO-PPO-PEO triblock polymer; poly(ethyleneglycol)-b-(2-(dimethylamino)ethyl methacrylate-co-methyl methacrylate) (PEG-b-(DMAEM-co-MMA); poly(methoxydiethylene glycol methacrylate (PmDEGMA); poly(methoxytriethylene glycol methacrylate (PmTEGMA); poly(N-isopropylacrylamide) (PNIPAM); PNIPAM-NH₂; poly(NIPAM-co-acrylic acid) (P(NIPAM-co-AA)); poly(NIPAM-acrylamide-allylamine); poly(NIPAM-co-N,N-dimethylacrylamide (P(NIPAM-co-N,NDMAM)); cholesterol-graft-poly[NIPAM-co-N-(hydroxymethyl) acrylamide] (CHOL-g-PNIPAM-co-NHMAAM); poly(NIPAM-co-N, NDMAM)-b-poly(D,L-lactide-co-glycolide) (P(NIPAM-co-N,NDMAM-b-PLGA); poly(NIPAM-co-N,NDMAM)-b-poly(lactide) (P(NIPAM-co-N,NDMAM-b-PLa); poly(NIPAM-co-acrylamide) (P(NIPAM-co-AAM)); poly(NIPAM-co-N,NDMAM)-b-poly (lactide-co-caprolactone) (P(NIPAM-co-N,NDMAM-b-P(LA-co-CL); poly[NIPAM-b-poly (butyl methacrylate)] (P(NIPAM-b-PBMA); Fe₃O₄-P(NIPAM); poly[monomethyl oligo (ethylene glycol)acrylate-2-(5,5-dimethyl-1,3-dioxan-2-yloxy) ethyl acrylate] (P(MOEGA-DMDEA); poly(N-vinylcaprolactam); a thermoplastic polyurethane; poly(2-isopropoyl-2-oxazoline); poly(-ethyl-2-oxazoline), poly(-nonyl-2-oxazoline); poly(L-valine-L-proline-L-glycine-X-L-glycine)ₙ where X is a neutral amino acid other than L-proline; poly(vinyl methyl ether); poly[tri(ethylene glycol) monoethyl ether methacrylate]; poly[2-(2-methoxyethoxy) ethyl methacrylate-co-oligo (ethylene glycol) methacrylate]; poly(methyl glycidyl ether); poly(ethyl glycidyl ether); poly(ethylene glycol)-coated hollow gold nanospheres (PEG-HAuNS); a polysaccharide; collagen; cyclodextrin; chitosan; chitosan-graft-polyethylenimine; elastin; 1,2-diplamitoyl-sn-glycero-3-phosphatidylcholine:dipalmitoyl phospatidylglycerol:1-stearoyl-2-hydroxy-sn-glycero-3-phosphocholine:poly(ethylene glycol) methyl ether (Mₙ2000):1,2,-distearoyl-sn-glycero3-phosphoethanolamine (DPPC:DPPG:MSPC:mPEG2000-DSPE); DPPC:MSPC:DSPE-PEG2000; DPPC:1,2-distearoyl-sn-glycero-phosphocholine:1,2-dipalmitoyl-3-trimethylammonium-propane: DSPE-polyethylene glycol (Mₙ2000) (DPPC:DSPC:DPTAP:DSPE:PEG2000); DPPC:monopalmitoyl phosphatidylcholine:dipalmitoyl-sn-glycero-3-phosphatidylethanolamine:PEG2000 (DPPC-MPPC-DPPE-PEG2000); DPPC:hydrogenated soy phosphatidylcholine-CHOL:DPPE:PEG2000 (DPPC:HSPC:CHOL:DPPE:PEG2000); DPPC:CHOL:DSPE:PEG2000:DSPE:PEG2000-folate; or any combination thereof.

Example 52. The sealing member of any example herein, particularly any one of examples 22-51, wherein the substrate material comprises a textile.

Example 53. The sealing member of any example herein, particularly any one of examples 22-52, wherein the substrate material comprises polyethylene terephthalate or polyethylene.

Example 54. The sealing member of any example herein, particularly any one of examples 22-53, wherein the substrate comprises a woven, braided, or knitted fabric.

Example 55. An implantable prosthesis, comprising a sealing member according to any example herein, particularly any one of examples 22-54.

Example 56. The implantable prosthesis of any example herein, particularly example 55, wherein the implantable prosthesis is a prosthetic heart valve comprising an annular frame and the sealing member is mounted outside or inside the annular frame.

Example 57. A method of making a sealing member, comprising: providing a substrate comprising a substrate material, the substrate having an outwardly facing surface and an inwardly facing surface, the substrate having a length L; and forming a hydrogel structure on the outwardly facing surface of the substrate, the hydrogel structure comprising a hydrogel.

Example 58. The method of any example herein, particularly example 57, wherein forming the hydrogel structure on the outwardly facing surface of the substrate comprises extruding or molding the hydrogel structure onto the outwardly facing surface of the substrate.

Example 59. The method of any example herein, particularly example 58, wherein the hydrogel structure is a linear structure extending across a width of the substrate.

Example 60. The method of any example herein, particularly example 59, wherein the linear structure has a zigzag configuration.

Example 61. The method of any example herein, particularly example 59 or example 60, further comprising forming a plurality of spaced-apart linear structures, each linear structure extending across the width of the substrate.

Example 62. The method of any example herein, particularly example 58, wherein the hydrogel structure is a plurality of spaced-apart discontinuous linear structures, each linear structure extending across a portion of a width of the substrate.

Example 63. The method of any example herein, particularly example 58, wherein the hydrogel structure is a plurality of spaced-apart linear structures extending along the length L of the substrate, each linear structure having a length L2 that is less than the length L of the substrate.

Example 64. The method of any example herein, particularly example 57, wherein forming the hydrogel structure on the outwardly facing surface of the substrate comprises mechanically attaching the hydrogel structure to the outwardly facing surface of the substrate.

Example 65. The method of any example herein, particularly example 64, wherein mechanically attaching the hydrogel structure to the outwardly facing surface of the substrate comprises: applying the hydrogel structure to the outwardly facing surface of the substrate; and ultrasonically attaching the hydrogel structure to the outwardly facing surface of the substrate.

Example 66. The method of any example herein, particularly example 64, wherein mechanically attaching the hydrogel structure to the outwardly facing surface of the substrate comprises: applying the hydrogel structure to the outwardly facing surface of the substrate, and applying pressure to adhere the hydrogel structure to the outwardly facing surface of the substrate.

Example 67. The method of any example herein, particularly any one of examples 64-66, wherein the hydrogel structure is a linear structure extending across a width of the substrate.

Example 68. The method of any example herein, particularly example 67, wherein the linear structure has a zigzag configuration.

Example 69. The method of any example herein, particularly example 67 or example 68, further comprising forming a plurality of spaced-apart linear structures, each linear structure extending across the width of the substrate.

Example 70. The method of any example herein, particularly any one of examples 64-66, wherein the hydrogel structure is a plurality of spaced-apart discontinuous linear structures, each linear structure extending across a portion of a width of the substrate.

Example 71. The method of any example herein, particularly any one of examples 64-66, wherein the hydrogel structure is a plurality of spaced-apart linear structures extending along the length L of the substrate, each linear structure having a length L2 that is less than the length L of the substrate.

Example 72. The method of any example herein, particularly example 57, wherein forming the hydrogel structure on the outwardly facing surface of the substrate comprises chemically attaching the hydrogel structure to the outwardly facing surface of the substrate.

Example 73. The method of any example herein, particularly example 72, wherein chemically attaching hydrogel structure on the outwardly facing surface of the substrate comprises: applying the hydrogel structure on the outwardly facing surface of the substrate; and crosslinking functional groups of the hydrogel to functional groups of the substrate material using ultraviolet irradiation.

Example 74. The method of any example herein, particularly example 72, wherein chemically attaching hydrogel structure on the outwardly facing surface of the substrate comprises: applying the hydrogel structure on the outwardly facing surface of the substrate; and hydrolyzing or oxidizing functional groups of the hydrogel, functional groups of the substrate material, or functional groups of the hydrogel and the substrate material, whereby functional groups of the hydrogel react with functional groups of the substrate material.

Example 75. The method of any example herein, particularly any one of examples 72-74, wherein the hydrogel structure is a linear structure extending across a width of the substrate.

Example 76. The method of any example herein, particularly example 75, wherein the linear structure has a zigzag configuration.

Example 77. The method of any example herein, particularly example 75 or example 76, further comprising forming a plurality of spaced-apart linear structures, each linear structure extending across the width of the substrate.

Example 78. The method of any example herein, particularly any one of examples 72-74, wherein the hydrogel structure is a plurality of spaced-apart discontinuous linear structures, each linear structure extending across a portion of a width of the substrate.

Example 79. The method of any example herein, particularly any one of examples 72-74, wherein the hydrogel structure is a plurality of spaced-apart linear structures extending along the length L of the substrate, each linear structure having a length L2 that is less than the length L of the substrate.

Example 80. The method of any example herein, particularly example 57, wherein forming the hydrogel structure on the outwardly facing surface of the substrate comprises dip-coating or spray-coating a hydrogel layer onto the outwardly facing surface of the substrate.

Example 81. The method of any example herein, particularly example 80, further comprising molding or etching the hydrogel layer to form the hydrogel structure.

Example 82. The method of any example herein, particularly example 81, further comprising using a laser to remove portions of the hydrogel layer to provide a first region having a first average thickness T1 that defines the hydrogel structure and adjacent regions having a second average thickness T2, wherein the second average thickness T2 is less than the first average thickness T1.

Example 83. The method of any example herein, particularly example 81 or example 82, wherein the hydrogel structure is a linear structure extending across a width of the substrate.

Example 84. The method of any example herein, particularly example 83, wherein the linear structure has a zigzag configuration.

Example 85. The method of any example herein, particularly example 83 or example 84, further comprising forming a plurality of spaced-apart linear structures, each linear structure extending across the width of the substrate.

Example 86. The method of any example herein, particularly example 81 or example 82, wherein the hydrogel structure is a plurality of spaced-apart discontinuous linear structures, each linear structure extending across a portion of a width of the substrate.

Example 87. The method of any example herein, particularly example 81 or example 82, wherein the hydrogel structure is a plurality of spaced-apart linear structures extending along the length L of the substrate, each linear structure having a length L2 that is less than the length L of the substrate.

Example 88. The method of any example herein, particularly any one of examples 57-87, further comprising mounting the sealing member around an outer surface of a frame of a prosthetic heart valve.

Example 89. method of any example herein, particularly any one of examples 57-88, wherein the hydrogel comprises a natural or synthetic hydrogel.

Example 90. The method of any example herein, particularly example 89, wherein the natural or synthetic hydrogel comprises a poloxamer, a poly(N-alkylacrylamide), a poly(n-vinylcaprolactam), a poly(alkyloxazoline), a poly(vinyl alkyl ether), a poly(alkyl glycidyl ether), a poly(methacrylic acid), a poly(alkylmethacrylate), a poly(acrylic acid), a poly(vinylpyridine), a poly(vinylimidazole), a poly(thiophene), a poly(alkyloxazoline),a polyamine, a sulfonated polystyrene, ethylene-vinyl acetate, polyurethane, poly(ethylene oxide) (PEO), poly(propylene oxide) (PPO), a polyketal, a polyacetal, a polylactide, a polyglycolide, a polysaccharide, collagen, a peptide, cyclodextrin, a nitrocatechol-terminated polymer, or any combination or copolymer thereof.

Example 91. The method of any example herein, particularly example 89, wherein the natural or synthetic hydrogel comprises a PEO-PPO co-block polymer; a PEO-PPO-PEO triblock polymer; poly(ethyleneglycol)-b-(2-(dimethylamino)ethyl methacrylate-co-methyl methacrylate) (PEG-b-(DMAEM-co-MMA); poly(methoxydiethylene glycol methacrylate (PmDEGMA); poly(methoxytriethylene glycol methacrylate (PmTEGMA); poly(N-isopropylacrylamide) (PNIPAM); PNIPAM-NH₂; poly(NIPAM-co-acrylic acid) (P(NIPAM-co-AA)); poly(NIPAM-acrylamide-allylamine); poly(NIPAM-co-N,N-dimethylacrylamide (P(NIPAM-co-N,NDMAM)); cholesterol-graft-poly[NIPAM-co-N-(hydroxymethyl) acrylamide] (CHOL-g-PNIPAM-co-NHMAAM); poly(NIPAM-co-N,NDMAM)-b-poly(D,L-lactide-co-glycolide) (P(NIPAM-co-N,NDMAM-b-PLGA); poly(NIPAM-co-N,NDMAM)-b-poly(lactide) (P(NIPAM-co-N,NDMAM-b-PLa); poly(NIPAM-co-acrylamide) (P(NIPAM-co-AAM)); poly(NIPAM-co-N,NDMAM)-b-poly(lactide-co-caprolactone) (P(NIPAM-co-N,NDMAM-b-P(LA-co-CL); poly[NIPAM-b-poly(butyl methacrylate)] (P(NIPAM-b-PBMA); Fe₃O₄-P(NIPAM); poly[monomethyl oligo (ethylene glycol)acrylate-2-(5,5-dimethyl-1,3-dioxan-2-yloxy) ethyl acrylate] (P(MOEGA-DMDEA); poly(N-vinylcaprolactam); a thermoplastic polyurethane; poly(-isopropoyl-2-oxazoline); poly(2-ethyl-2-oxazoline), poly(2-nonyl-2-oxazoline); poly(L-valine-L-proline-L-glycine-X-L-glycine)ₙ where X is a neutral amino acid other than L-proline; poly(vinyl methyl ether); poly[tri(ethylene glycol) monoethyl ether methacrylate]; poly[2-(2-methoxyethoxy) ethyl methacrylate-co-oligo (ethylene glycol) methacrylate]; poly(methyl glycidyl ether); poly(ethyl glycidyl ether); poly(ethylene glycol)-coated hollow gold nanospheres (PEG-HAuNS); a polysaccharide; collagen; cyclodextrin; chitosan; chitosan-graft-polyethylenimine; elastin; 1,2-diplamitoyl-sn-glycero-3-phosphatidylcholine:dipalmitoyl phospatidylglycerol:1-stearoyl-2-hydroxy-sn-glycero-3-phosphocholine:poly(ethylene glycol) methyl ether (Mₙ2000):1,2,-distearoyl-sn-glycero3-phosphoethanolamine (DPPC:DPPG:MSPC:mPEG2000-DSPE); DPPC:MSPC:DSPE-PEG2000; DPPC:1,2-distearoyl-sn-glycero-phosphocholine: 1,2-dipalmitoyl-3-trimethylammonium-propane: DSPE-polyethylene glycol (Mₙ2000) (DPPC:DSPC:DPTAP:DSPE:PEG2000); DPPC:monopalmitoyl phosphatidylcholine:dipalmitoyl-sn-glycero-3-phosphatidylethanolamine:PEG2000 (DPPC-MPPC-DPPE-PEG2000); DPPC:hydrogenated soy phosphatidylcholine-CHOL:DPPE:PEG2000 (DPPC:HSPC:CHOL:DPPE:PEG2000); DPPC:CHOL:DSPE:PEG2000:DSPE:PEG2000-folate; or any combination thereof.

Example 92. The method of any example herein, particularly any one of examples 57-91, wherein the substrate material comprises a textile.

Example 93. The method of any example herein, particularly any one of examples 57-92, wherein the substrate material comprises polyethylene terephthalate or polyethylene.

Example 94. The method of any example herein, particularly any one of examples 57-93, wherein the substrate comprises a woven, braided, or knitted fabric.

### V. Example

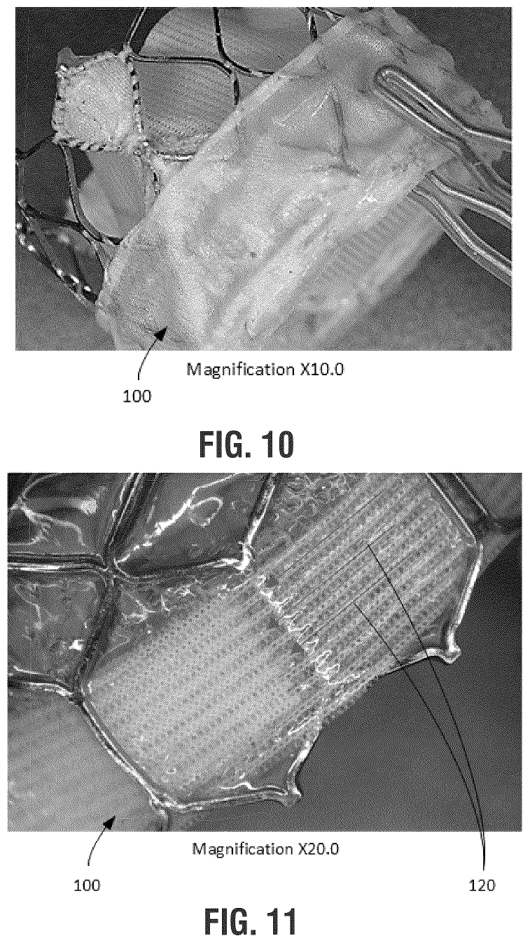

A thermoplastic polyurethane hydrogel (Tecophilic™ TPU, available from Lubrizol, Avon Lake, OH) was applied, by dip coating with masking, to a knitted outer skirt of a transcatheter heart valve. Tecophilic™ TPU expands and changes stiffness at 37° C. As shown in FIG. 10, the hydrogel did not impact the valve's overall dimensions at room temperature. A magnified view (FIG. 11) shows the hydrogel ridges on the knit warp skirt.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A sealing member for an implantable prosthesis, the sealing member comprising:
a substrate comprising a substrate material, the substrate having an outwardly facing surface and an inwardly facing surface; and
a stimulus-responsive hydrogel disposed on the outwardly facing surface of the substrate.

2. The sealing member of claim 1, wherein the stimulus-responsive hydrogel undergoes a change in volume, stiffness, or both induced by a stimulus, the stimulus comprising a temperature change, a pH change, an ionic strength change, a solvent composition change, application of an electric field, application of a magnetic field, application of ultrasound, exposure to light, or any combination thereof.

3. The sealing member of claim 1, wherein the stimulus-responsive hydrogel, in an ex vivo environment, has a first non-expanded thickness T1 measured from the outwardly facing surface of the substrate to an outer surface of the stimulus-responsive hydrogel, particularly wherein the stimulus-responsive hydrogel, in an in vivo environment, has a second expanded thickness T2, wherein T2 is greater than T1.

4. The sealing member of claim 1, wherein the sealing member comprises an outer skirt, the outer skirt comprising:
the substrate, wherein the substrate has a length L; and
the hydrogel structure disposed on the outwardly facing surface of the substrate.

5. The sealing member of claim 4, wherein a top width W_{T} of the hydrogel structure at the outer surface, in the in vivo environment, is 0.2 mm to 0.4 mm, particularly wherein the second expanded thickness T2 is from 10×W_{T} to 12×W_{T}.

6. The sealing member of claim 4, wherein:
(i) the hydrogel structure is an annular hydrogel ring; or
(ii) the hydrogel structure comprises a plurality of annular rings; or
(iii) the hydrogel structure comprises a plurality of spaced-apart axially extending hydrogel structures having a length L2 less than the length L of the substrate; or
(iv) the hydrogel structure comprises a plurality of spaced-apart hydrogel structures comprising a plurality of discontinuous circumferential structures, each hydrogel structure extending across a portion of the substrate.

7. The sealing member of claim 6, wherein the hydrogel structure is an annular hydrogen ring, and wherein:
(i) the annular hydrogen ring has a base width W_{B} that is less than the length L of the substrate, particularly wherein the base width W_{B} of the hydrogel structure is greater than or equal to a top width W_{T} of the hydrogel structure at an outer surface of the hydrogel structure; or
(ii) the hydrogel structure tapers from the base width W_{B} to the top width W_{T} at an angle α of 0 degrees to 20 degrees; or
(iii) the annular hydrogel ring has a zig zag configuration; or
(iv) the hydrogel structure comprises a plurality of annular rings, and a pitch p of the plurality of annular hydrogel rings is from 10×W_{T} to 18×W_{T}; or
(v) any combination of (i), (ii), (iii), and (iv).

8. The sealing member of claim 6, wherein:
the hydrogel structure comprises a plurality of spaced-apart axially extending hydrogel structures and each of the plurality of spaced-apart axially extending hydrogel structures has a base width W_{B} that is greater than or equal to a top width W_{T} at an outer surface of the hydrogel structure, particularly wherein each of the plurality of spaced-apart axially extending hydrogel structures tapers from the base width W_{B} to the top width W_{T} at an angle α of 0 degrees to 20 degrees; or the hydrogel structure comprises a plurality of discontinuous circumferential structures, each hydrogel structure having a base width W_{B} that is less than the length L of the substrate, wherein the base width W_{B} is greater than or equal to a top width W_{T} of the hydrogel structure at an outer surface of the hydrogel structure, particularly wherein each hydrogel structure tapers from the base width W_{B} to the top width W_{T} at an angle α of 0 degrees to 20 degrees.

9. The sealing member of claim 4, wherein the hydrogel structure is a layer of the stimulus-responsive hydrogel disposed on the outwardly facing surface of the substrate.

10. The sealing member of claim 9, wherein the layer of the stimulus-responsive hydrogel comprises regions of a first average thickness T3 defining a plurality of spaced-apart hydrogel structures alternating with regions of a second average thickness T4, wherein the first average thickness T3 is greater than the second average thickness T4, and wherein:
the plurality of spaced-apart hydrogel structures is a plurality of spaced-apart annular hydrogel rings; or
the plurality of spaced-apart hydrogel structures is a plurality of spaced-apart annular hydrogel rings, wherein each of the plurality of spaced-apart annular hydrogel rings has a zigzag configuration; or
the plurality of spaced-apart hydrogel structures comprises a plurality of spaced-apart axially extending hydrogel structures having a length L2 less than the length L of the substrate; or
the plurality of spaced-apart hydrogel structures comprises a plurality of discontinuous circumferential structures, each hydrogel structure extending around a portion of the substrate.

11. The sealing member of claim 1, wherein:
(i) the substrate material comprises polyethylene terephthalate or polyethylene; or
(ii) the substrate comprises a woven, braided, or knitted fabric; or
(iii) both (i) and (ii).

12. A prosthetic heart valve comprising:
an annular frame configured to be radially compressible and expandable between a radially compressed state and a radially expanded state;
a valvular structure disposed within the annular frame and configured to regulate flow of blood through the annular frame in portions; and
a sealing member comprising a fabric substrate and a hydrogel structure attached to the fabric substrate, the hydrogel structure comprising a stimulus-responsive hydrogel and having an exposed outer surface configured to seal against tissue surrounding the prosthetic heart valve when implanted in a patient's body.

13. The prosthetic heart valve of claim 12, wherein the sealing member comprises:
an outer skirt that extends around an outer surface of the annular frame; or
an inner skirt that extends along an inner surface of the annular frame.

14. The prosthetic heart valve of claim 12, wherein the stimulus-responsive hydrogel undergoes a change in volume, stiffness, or both induced by a stimulus, the stimulus comprising a temperature change, a pH change, an ionic strength change, a solvent composition change, application of an electric field, application of a magnetic field, application of ultrasound, exposure to light, or any combination thereof.

15. The prosthetic heart valve of claim 12, wherein:
(i) the hydrogel structure is bonded to the fabric substrate; or
(ii) the fabric substrate comprises polyethylene terephthalate or polyethylene; or
(iii) the fabric substrate comprises a woven, braided, or knitted fabric; or
(iv) any combination of (i), (ii), and (iii).

16. The prosthetic heart valve of claim 12, wherein the stimulus-responsive hydrogel comprises a natural or synthetic hydrogel,
particularly wherein the natural or synthetic hydrogel comprises a poloxamer, a poly(N-alkylacrylamide), a poly(n-vinylcaprolactam), a poly(alkyloxazoline), a poly(vinyl alkyl ether), a poly(alkyl glycidyl ether), a poly(methacrylic acid), a poly(alkylmethacrylate), a poly(acrylic acid), a poly(vinylpyridine), a poly(vinylimidazole), a poly(thiophene), a poly(alkyloxazoline),a polyamine, a sulfonated polystyrene, ethylene-vinyl acetate, polyurethane, poly(ethylene oxide) (PEO), poly(propylene oxide) (PPO), a polyketal, a polyacetal, a polylactide, a polyglycolide, a polysaccharide, collagen, a peptide, cyclodextrin, a nitrocatechol-terminated polymer, or any combination or copolymer thereof.

17. The prosthetic heart valve of claim 16, wherein the natural or synthetic hydrogel comprises:
a PEO-PPO co-block polymer;
a PEO-PPO-PEO triblock polymer;
poly(ethyleneglycol)-b-(2-(dimethylamino)ethyl methacrylate-co-methyl methacrylate) (PEG-b-(DMAEM-co-MMA);
poly(methoxydiethylene glycol methacrylate (PmDEGMA);
poly(methoxytriethylene glycol methacrylate (PmTEGMA);
poly(N-isopropylacrylamide) (PNIPAM);
PNIPAM-NH₂;
poly(NIPAM-co-acrylic acid) (P(NIPAM-co-AA));
poly(NIPAM-acrylamide-allylamine);
poly(NIPAM-co-N,N-dimethylacrylamide (P(NIPAM-co-N,NDMAM));
cholesterol-graft- poly[NIPAM-co-N-(hydroxymethyl) acrylamide] (CHOL-g-PNIPAM-co-NHMAAM);
poly(NIPAM-co-N,NDMAM)-b-poly(D,L-lactide-co-glycolide) (P(NIPAM-co-N,NDMAM-b-PLGA);
poly(NIPAM-co-N,NDMAM)-b-poly(lactide) (P(NIPAM-co-N,NDMAM-b-PLa);
poly(NIPAM-co-acrylamide) (P(NIPAM-co-AAM));
poly(NIPAM-co-N,NDMAM)-b-poly(lactide-co-caprolactone) (P(NIPAM-co-N,NDMAM-b-P(LA-co-CL);
poly[NIPAM-b-poly(butyl methacrylate)] (P(NIPAM-b-PBMA);
Fe₃O₄-P(NIPAM);
poly[monomethyl oligo (ethylene glycol)acrylate-2-(5,5-dimethyl-1,3-dioxan-2-yloxy) ethyl acrylate] (P(MOEGA-DMDEA);
poly(N-vinylcaprolactam);
a thermoplastic polyurethane;
poly(-isopropoyl-2-oxazoline);
poly(-ethyl-2-oxazoline), poly(-nonyl-2-oxazoline);
poly(L-valine-L-proline-L-glycine-X-L-glycine)ₙ where X is a neutral amino acid other than L-proline;
poly(vinyl methyl ether);
poly[tri(ethylene glycol) monoethyl ether methacrylate];
poly[2-(2-methoxyethoxy) ethyl methacrylate-co-oligo (ethylene glycol) methacrylate];
poly(methyl glycidyl ether);
poly(ethyl glycidyl ether);
poly(ethylene glycol)-coated hollow gold nanospheres (PEG-HAuNS);
a polysaccharide;
collagen;
cyclodextrin;
chitosan;
chitosan-graft-polyethylenimine;
elastin;
1,2-diplamitoyl-sn-glycero-3-phosphatidylcholine:dipalmitoyl phospatidylglycerol:1-stearoyl-2-hydroxy-sn-glycero-3-phosphocholine:poly(ethylene glycol) methyl ether (Mₙ2000):1,2,-distearoyl-sn-glycero3-phosphoethanolamine (DPPC:DPPG:MSPC:mPEG2000-DSPE);
DPPC:MSPC:DSPE-PEG2000;
DPPC:1,2-distearoyl-sn-glycero-phosphocholine:1,2-dipalmitoyl-3-trimethylammonium-propane: DSPE-polyethylene glycol (Mₙ2000) (DPPC:DSPC:DPTAP:DSPE:PEG2000);
DPPC:monopalmitoyl phosphatidylcholine:dipalmitoyl-sn-glycero-3-phosphatidylethanolamine: PEG2000 (DPPC-MPPC-DPPE-PEG2000);
DPPC:hydrogenated soy phosphatidylcholine-CHOL:DPPE:PEG2000 (DPPC:HSPC:CHOL:DPPE:PEG2000);
DPPC:CHOL:DSPE:PEG2000:DSPE:PEG2000-folate; or any combination thereof.

18. A method of making a sealing member, comprising:
providing a substrate comprising a substrate material, the substrate having an outwardly facing surface and an inwardly facing surface, the substrate having a length L; and
forming a hydrogel structure on the outwardly facing surface of the substrate, the hydrogel structure comprising a hydrogel, particularly wherein forming the hydrogel structure on the outwardly facing surface of the substrate comprises extruding or molding the hydrogel structure onto the outwardly facing surface of the substrate.

19. The method of claim 18, wherein forming the hydrogel structure on the outwardly facing surface of the substrate comprises:
extruding or molding the hydrogel structure onto the outwardly facing surface of the substrate; or
mechanically attaching the hydrogel structure to the outwardly facing surface of the substrate; or
chemically attaching the hydrogel structure to the outwardly facing surface of the substrate; or
dip-coating or spray-coating a hydrogel layer onto the outwardly facing surface of the substrate.
